(19) United States
(12) Patent Application Publication
(10) Pub. No.: US 2013/0040281 A1
(43) Pub. Date: Feb. 14, 2013

(54) Procurement of Placental Stem Cells

(71) Applicant: Robert A. Dracker, Liverpool, NY (US)

(72) Inventor: Robert A. Dracker, Liverpool, NY (US)

(21) Appl. No.: 13/584,135

(22) Filed: Aug. 13, 2012

Related U.S. Application Data
(60) Provisional application No. 61/522,287, filed on Aug. 11, 2011.

(51) Int. Cl. C12N 5/071; A01N 1/02

(52) U.S. Cl. 435/1.2

(57) ABSTRACT

Methods and solutions for the preservation and procurement of placental stem cells. A method for collecting stem cells from a placenta comprises: draining cord blood from the placenta; perfusing the drained placenta with a perfusion solution comprising 1,1′-[1,4-phenylenebis(methylene)]-bis-1,4,8,11-tetraazacyclotetradecane or a pharmaceutically acceptable salt thereof, an aminoglycoside antibiotic, papaverine, and a placental preservative; and collecting the stem cells and perfusion solution from the placenta.

### CROSS-REFERENCE TO RELATED APPLICATIONS

This application claims priority to U.S. Provisional Patent Application Ser. No. 61/522,287, filed on Aug. 11, 2011 and entitled “Procurement of Placental Stem Cells,” the entire disclosure of which is incorporated herein by reference.

### BACKGROUND OF THE INVENTION

1. Field of the Invention

The present invention relates to placental stem cells and, more specifically, to a method for the procurement of placental stem cells.

2. Description of the Related Art

Stem cells are master cells found in all multicellular organisms. These special cells are important to the human body, for example, because they are capable of: (i) differentiating into a multitude of different specialized cell types; and (ii) dividing to maintain a supply of stem cells. In humans there are two main types of stems cells: embryonic stem cells and adult stem cells. In a developing embryo stem cells differentiate into all types of cells, thereby creating specialized tissues, organs, and systems. In an adult human, stem cells are involved in the normal turnover of organs such as blood and skin.

Hematopoietic stem cells, for example, are used to treat blood and immune system diseases because they can differentiate into red blood cells, white blood cells, and platelets. However, some stem cell transplants have been performed for patients with genetic or metabolic diseases. Indeed, to date more than 80 different diseases have been treated using stem cell transplants. According to the National Cord Blood Program, there were over 15,000 through the end of 2009. The National Marrow Donor Program estimates that there will be 10,000 cord blood transplants per year by 2015, up from 2,000 per year in 2006.

In addition to known treatments involving stem cells, research continues into the promise of many potential future applications. Indeed, the ability of stem cells to differentiate into other types of cells holds significant promise for treating some of the world's most common diseases including heart disease, diabetes, stroke, hearing loss, blood disorders, Parkinson's disease, and Alzheimer's disease, just to name a few.

Umbilical cord blood—blood which remains in the placenta and umbilical cord after childbirth—is one of the most common sources of stem cells. Since cord blood is collected from the placenta, which is normally discarded, the collection process is safe for both the mother and the newborn.

Cord blood is obtained by syringing out the placenta through the umbilical cord shortly after childbirth, after the cord has been detached from the newborn. The retrieved blood can then be frozen and stored indefinitely.

Although the amount of stem cells obtained from cord blood is generally enough to treat a child, there are generally not enough stem cells to treat an adult patient. The placenta is a better source of stem cells, since it can contain up to ten times more stem cells than cord blood. Still, even when blood is retrieved from both the umbilical cord and placenta using current collection methods, the amount of stem cells is often not suitable to treat an adult patient.

As a result, there is a continued need for cord blood collection methods and devices that significantly increase the number of stem cells collected, facilitate the collection of stem cells, or allow for the collection of enough stem cells to treat at least one adult patient.

### BRIEF SUMMARY OF THE INVENTION

It is therefore a principal object and advantage of the present invention to provide a method, device, and/or system for the collection of cord blood.

It is another object and advantage of the present invention to provide a method, device, and/or system to increase the number of stem cells collected from cord blood.

Other objects and advantages of the present invention will in part be obvious, and in part appear hereinafter.

Methods and solutions for the preservation and procurement of placental stem cells. According to one aspect, a method for collecting stem cells from a placenta comprises: draining cord blood from the placenta; perfusing the drained placenta with a perfusion solution, the perfusion solution comprising a placental preservative, a stem cell releasing agent, an antibiotic, and a vasodilator; and collecting the stem cells and perfusion solution from the placenta.

In one implementation, the placental preservative comprises NaCl, KCl, glucose, citric acid, adenine, histidine, glutamate, glutathione, and N-acetyl-L-cysteine.

In one implementation, the placental preservative comprises NaCl at a concentration of approximately 4.3 g/L, KCl at a concentration of approximately 0.45 g/L, glucose at a concentration of approximately 1 g/L, citric acid at a concentration of approximately 2.2 g/L, adenine at a concentration of approximately 0.25 g/L, histidine at a concentration of approximately 4.2 g/L, glutamate at a concentration of approximately 1.9 g/L, glutathione at a concentration of approximately 0.92 g/L, and N-acetyl-L-cysteine at a concentration of approximately 0.016 g/L.

In one implementation, the placental preservative comprises approximately 125 to 150 mM Na⁺; approximately 5 to 7 mM K⁺; approximately 50 to 150 mM Cl⁻; and/or approximately 280 to 300 mOsm/kg.

In one implementation, the stem cell releasing agent is 1,1′-[1,4-phenylenebis (methylene)]-bis-1,4,8,11-tetraazacyclotetradecane or a pharmaceutically acceptable salt thereof.

In one implementation, the antibiotic comprises an aminoglycoside antibiotic including but not limited to gentamicin. In one implementation, gentamicin is administered at a concentration of approximately 10 mg/100 ml.

In one implementation, the vasodilator comprises papaverine. In one implementation, the papaverine is administered at a concentration of approximately 100 mg/100 ml.

In one implementation, the vasodilator is selected from the group consisting of nitroglycerine, a Ca²⁺ channel blocker, an opium alkaloid, and combinations thereof.

In another aspect, a method for collecting stem cells from a placenta comprises: draining draining cord blood from the placenta; perfusing the drained placenta with a perfusion solution, the perfusion solution comprising 1,1′-[1,4-phenylenebis (methylene)]-bis-1,4,8,11-tetraazacyclotetradecane or a pharmaceutically acceptable salt thereof, an aminoglycoside antibiotic, papaverine, and a placental preservative, the placental preservative comprising NaCl, KCl, glucose, citric acid, adenine, histidine, glutamate, glutathione, and N-acetyl-L-cysteine; and collecting the stem cells and perfusion solution from the placenta.

In one implementation, the placental preservative comprises NaCl at a concentration of approximately 4.3 g/L, KCl at a concentration of approximately 0.45 g/L, glucose at a concentration of approximately 1 g/L, citric acid at a concentration of approximately 2.2 g/L, adenine at a concentration of approximately 0.25 g/L, histidine at a concentration of approximately 4.2 g/L, glutamate at a concentration of approximately 1.9 g/L, glutathione at a concentration of approximately 0.92 g/L, and N-acetyl-L-cysteine at a concentration of approximately 0.016 g/L.

In another aspect, a placental perfusion solution comprising: a placental preservative; a stem cell releasing agent; an antibiotic; and a vasodilator.

In one implementation, the stem cell releasing agent comprises 1,1′-[1,4-phenylenebis(methylene)]-bis-1,4,8,11-tetraazacyclotetradecane, said antibiotic comprises an aminoglycoside antibiotic, and said vasodilator comprises papaverine.

In one implementation, the placental preservative comprises NaCl, KCl, glucose, citric acid, adenine, histidine, glutamate, glutathione, and N-acetyl-L-cysteine.

In one implementation, the placental preservative NaCl at a concentration of approximately 4.3 g/L, KCl at a concentration of approximately 0.45 g/L, glucose at a concentration of approximately 1 g/L, citric acid at a concentration of approximately 2.2 g/L, adenine at a concentration of approximately 0.25 g/L, histidine at a concentration of approximately 4.2 g/L, glutamate at a concentration of approximately 1.9 g/L, glutathione at a concentration of approximately 0.92 g/L, and N-acetyl-L-cysteine at a concentration of approximately 0.016 g/L.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a flowchart of a method of stem cell collection according to one embodiment of the present invention.

### DETAILED DESCRIPTION OF THE INVENTION

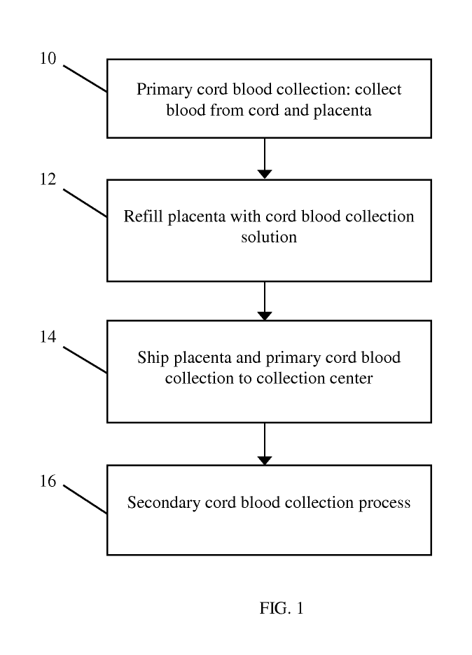

Referring now to the drawings, wherein like reference numerals refer to like parts throughout, there is seen in FIG. 1 a flowchart of a method of stem cell collection according to one embodiment of the present invention. At step 10 is the primary cord blood collection procedure. This is accomplished using any of a variety of known cord blood collection methods, or any method capable of collecting the excess cord blood from the umbilical cord and/or placenta shortly after childbirth. For example, the cord blood can be collected using the following procedure: (i) deliver the baby vaginally or via cesarean section; (ii) clamp the umbilical cord at both the mother's and the child's side and cut the cord at a location between the clamps; (iii) insert a tube or needle into the umbilical vein to collect the blood via gravity; and (iv) ship the blood off for processing and dispose of the placenta. In a preferred embodiment, the cord blood is collected after delivery of the baby but before delivery of the placenta.

As described in the above cord blood collection method, the placenta is typically disposed and/or destroyed upon completion of the typical cord blood collection procedure. As such, these methods do not proceed past step 10 of the present method.

At step 12 of the present method, the placenta is filled with the cord blood collection solution. According to one embodiment of the present invention, the cord blood collection solution comprises the following:
i. a placental preservative base;
ii. a stem cell releasing agent;
iii. an antibiotic; and
iv. a vasodilator.

According to one embodiment of the present invention, the stem cell releasing agent can be any agent capable of causing stem cells to release from normal storage sites into the blood. According to one embodiment of the present invention, the stem cell releasing agent is AMD3100 1,1′-[1,4-Phenylenebis(methylene)]bis[1,4,8,11-tetraazacyclotetradecane] (or a pharmaceutically acceptable salt thereof), also known by the International Nonproprietary Name plexifor, by the trade name MOZOBIL®, and as JM 3100), preferably at a concentration of approximately 3-10 mg per 100 ml, although other concentrations are possible. The AMD3100 causes, among other conditions, the release of stem cells from the placenta blood vessel walls, resulting in substantially higher amounts of stem cells to be collected via the process described herein. The antibiotic can be, for example, any agent capable of providing bactericidal properties against a range of human bacterial infections, while being substantially non-toxic to humans. According to one implementation, the antibiotic is an aminoglycoside antibiotic. One such antibiotic is gentamicin, which is preferably at a concentration of approximately 10 mg per 100 ml in this embodiment, although other concentrations are possible. Lastly, the vasodilator can be any agent capable of dilating the blood vessels of the umbilical cord and placenta. One such vasodilator is papaverine, which is preferably at a concentration of approximately 100 mg per 100 ml in this embodiment, although other concentrations are possible. Other vasodilators include but are not limited to nitroglycerine, Ca²⁺ channel blockers, and other opium alkaloids, for example.

The placental preservative base can be any solution capable of preserving the placenta during shipping and short- and/or long-term storage. According to one embodiment of the present invention, the placental preservative base comprises the following:
i. NaCl (4.3 g/L or 74 mM);
ii. KCl (0.45 g/L or 6 mM);
iii. D-glucose (1 g/L or 5.6 mM);
iv. citric acid anhydrous (2.2 g/L or 15 mM);
v. adenine free base (0.25 g/L or 1.85 mM);
vi. histidine (4.2 g/L or 20 mM);
vii. glutamate (1.9 g/L or 13 mM)
viii. glutathione (0.92 g/L or 3 mM; and
ix. N-acetyl-L-cysteine (also known as acetylcysteine or N-acetylcysteine) (0.016 g/L or 0.1 mM).

Although a preferred concentration is provided, one of ordinary skill in the art would recognize that one or more of the above concentrations can be varied without affecting the effectiveness or activity of the placental preservative base. Further, one of ordinary skill in the art would recognize that one or more of the above components can be omitted or replaced depending on a variety of factors. According to one embodiment, the target electrolyte and glucose profile of the placental preservative base comprises: (i) approximately 0 to 250 mM Na⁺, with a preferred embodiment comprising approximately 125 to 150 mM Na⁺; (ii) approximately 0 to 25 mM K⁺, with a preferred embodiment comprising approximately 5 to 7 mM K⁺; (iii) approximately 0 to 250 mM Cl⁻, with a preferred embodiment comprising approximately 50 to 150 mM Cl⁻; (iv) 0 to 10 mM glucose, with a preferred embodiment comprising approximately 5 mM glucose; (v) and osmolarity of 100 to 400 mOsm/kg, with a preferred embodiment comprising approximately 280 to 300 mOsm/kg.

In a preferred embodiment, the cord blood collection solution is allowed to fill the placenta via gravity through a needle or tube inserted into the umbilical vein and/or other locations in the placenta. Once the placenta contains a sufficient amount of cord blood collection solution, decided by a variety of factors including the predetermined amount of solution provided for collection, the size of the placenta, and/or prescribed collection procedure, among many others, the umbilical cord is once again clamped shut such that the injected solution remains inside the placenta.

Once the placenta has been refilled with the cord blood collection solution at step 12, the placenta is processed for shipping to a processing/collection center. This can include, among other things, processing the umbilical cord to ensure clamping, placing the placenta in a container suitable for short- or long-term shipping or storage, and/or freezing the placenta. Together with the primary cord blood collection, the placenta is then shipped or delivered to the processing/collection center. At some locations, the processing/collection center may be the same institution or near the same institution as the baby delivery center.

At step 16, the placenta undergoes processing at the processing/collection center. The cord blood collection solution is collected from the placenta at this step. This “secondary collection” is then processed to concentrate the mononuclear cell content and is then cryogenically stored together, but in separate containers, with the primary cord collection. The two collections may or may not be combined prior to storage depending upon a variety of factors, including but not limited to storage limitations and known or possible future uses of the stem cells, among others. The placenta can be cryogenically stored as well, or can be disposed of once the procedure is complete. In a preferred embodiment, the cord blood collection solution is allowed to drain from the placenta via gravity through a needle or tube inserted into the umbilical vein and/or other locations in the placenta. To begin draining, the umbilical cord is unclamped to allow the egress of fluid, and any drained fluid is then collected.

In yet another embodiment, the placenta is cryogenically stored prior to the secondary collection. In this embodiment, the injected cord blood collection solution is stored inside the placenta until it is needed, or until it can be more properly processed.

Although the present invention has been described in connection with a preferred embodiment, it should be understood that modifications, alterations, and additions can be made to the invention without departing from the scope of the invention as defined by the claims.

What is claimed is::

1. A method for collecting stem cells from a placenta, the method comprising:
draining cord blood from the placenta;
perfusing the drained placenta with a perfusion solution, the perfusion solution comprising a placental preservative, a stem cell releasing agent, an antibiotic, and a vasodilator; and
collecting the stem cells and perfusion solution from the placenta.

2. The method of claim 1, wherein the placental preservative comprises NaCl, KCl, glucose, citric acid, adenine, histidine, glutamate, glutathione, and N-acetyl-L-cysteine.

3. The method of claim 2, wherein the placental preservative comprises NaCl at a concentration of approximately 4.3 g/L, KCl at a concentration of approximately 0.45 g/L, glucose at a concentration of approximately 1 g/L, citric acid at a concentration of approximately 2.2 g/L, adenine at a concentration of approximately 0.25 g/L, histidine at a concentration of approximately 4.2 g/L, glutamate at a concentration of approximately 1.9 g/L, glutathione at a concentration of approximately 0.92 g/L, and N-acetyl-L-cysteine at a concentration of approximately 0.016 g/L.

4. The method of claim 1, wherein the placental preservative comprises approximately 125 to 150 mM Na⁺.

5. The method of claim 1, wherein the placental preservative comprises approximately 5 to 7 mM K⁺.

6. The method of claim 1, wherein the placental preservative comprises approximately 50 to 150 mM Cl⁻.

7. The method of claim 1, wherein osmolarity of the placental preservative is approximately 280 to 300 mOsm/kg.

8. The method of claim 1, wherein the stem cell releasing agent is 1,1′-[1,4-phenylenebis(methylene)]-bis-1,4,8,11-tetraazacyclotetradecane or a pharmaceutically acceptable salt thereof.

9. The method of claim 1, wherein the antibiotic comprises an aminoglycoside antibiotic.

10. The method of claim 10, wherein the antibiotic comprises gentamicin.

11. The method of claim 11, wherein the gentamicin is administered at a concentration of approximately 10 mg/100 ml.

12. The method of claim 1, wherein the vasodilator comprises papaverine.

13. The method of claim 12, wherein the papaverine is administered at a concentration of approximately 100 mg/100 ml.

14. The method of claim 1, wherein the vasodilator is selected from the group consisting of nitroglycerine, a Ca²⁺ channel blocker, an opium alkaloid, and combinations thereof.

15. A method for collecting stem cells from a placenta, the method comprising:
draining cord blood from the placenta;
perfusing the drained placenta with a perfusion solution, the perfusion solution comprising 1,1′-[1,4-phenylenebis(methylene)]-bis-1,4,8,11-tetraazacyclotetradecane or a pharmaceutically acceptable salt thereof, an aminoglycoside antibiotic, papaverine, and a placental preservative, the placental preservative comprising NaCl, KCl, glucose, citric acid, adenine, histidine, glutamate, glutathione, and N-acetyl-L-cysteine; and
collecting the stem cells and perfusion solution from the placenta.

16. The method of claim 15, wherein the placental preservative comprises NaCl at a concentration of approximately 4.3 g/L, KCl at a concentration of approximately 0.45 g/L, glucose at a concentration of approximately 1 g/L, citric acid at a concentration of approximately 2.2 g/L, adenine at a concentration of approximately 0.25 g/L, histidine at a concentration of approximately 4.2 g/L, glutamate at a concentration of approximately 1.9 g/L, glutathione at a concentration of approximately 0.92 g/L, and N-acetyl-L-cysteine at a concentration of approximately 0.016 g/L.

17. A placental perfusion solution comprising:
a placental preservative;
a stem cell releasing agent;
an antibiotic;
and a vasodilator.

18. The placental perfusion solution of claim 17, wherein said stem cell releasing agent comprises 1,1′-[1,4-phenylenebis(methylene)]-bis-1,4,8,11-tetraazacyclotetradecane, said antibiotic comprises an aminoglycoside antibiotic, and said vasodilator comprises papaverine.

19. The placental perfusion solution of claim 17, wherein said placental preservative comprises NaCl, KCl, glucose, citric acid, adenine, histidine, glutamate, glutathione, and N-acetyl-L-cysteine.

20. The placental perfusion solution of claim 17, wherein said placental preservative NaCl at a concentration of approximately 4.3 g/L, KCl at a concentration of approximately 0.45 g/L, glucose at a concentration of approximately 1 g/L, citric acid at a concentration of approximately 2.2 g/L, adenine at a concentration of approximately 0.25 g/L, histidine at a concentration of approximately 4.2 g/L, glutamate at a concentration of approximately 1.9 g/L, glutathione at a concentration of approximately 0.92 g/L, and N-acetyl-L-cysteine at a concentration of approximately 0.016 g/L.
